(19) United States
(12) Patent Application Publication
(10) Pub. No.: US 2021/0046211 A1
(43) Pub. Date: Feb. 18, 2021

(54) Ceramic Part Having At Least One Ceramic Foam for Medical Applications

(71) Applicant: CeramTec GmbH, Plochingen (DE)

(72) Inventors: Ulrike DEISINGER, Erlangen (DE); Michael GÖTZ, Uttenreuth (DE); Andreas REMPP, Ostfildern (DE)

(21) Appl. No.: 16/641,001

(22) PCT Filed: Aug. 14, 2018
(86) PCT No.: PCT/EP2018/072058

§ 371 Date: Feb. 21, 2020

(30) Foreign Application Priority Data
Aug. 25, 2017 (DE) 10 2017 214 886.1

(51) Int. Cl. A61L 27/10; A61L 27/42; A61L 27/56; C04B 35/119; C04B 35/488; C04B 35/587; C04B 35/565; C04B 38/02

(52) U.S. Cl. CPC A61L 27/10; A61L 27/427; A61L 27/56; C04B 35/119; C04B 2111/00836; C04B 35/587; C04B 35/565; C04B 38/02; A61L 2400/18; C04B 35/4885

(57) ABSTRACT

The invention relates to the use of ceramic parts that at least partly consist of a ceramic foam in the field of medical technology.

The invention relates to the use of ceramic parts, preferably in the field of medical technology, with the parts being formed at least in part by a ceramic foam. In the prior art, ceramic components that comprise at least one porous part or consist entirely of a porous ceramic material are known in the field of medical technology, for example in the field of implant technology. In general, a wide range of processes and methods are known for producing the porous structures. These include, for example, slip-based methods, in which ceramic porous structures on components or entire porous components can be produced by means of a ceramic slip containing organic, structural pore-forming agents or chemical substances. The ceramic slips can be understood to be suspensions that include a liquid medium, a ceramic starting powder, and optionally additional additives.

However, the problem with the thus produced ceramic components, in particular implants, which comprise or consist of a porous part, is that the metal-free porous structures often only have low stability and in particular can only be poorly machined during operations. The insertions of screws or nails, for example for temporarily fixing the ceramic component, may result in a catastrophic failure of the porous structure or the entire implant in porous structures produced by means of known methods.

The problem addressed by the present invention was thus to provide ceramic parts that can be used in medical technology which consist of a ceramic foam at least in part or even entirely and do not have the drawbacks of the known components. In particular, ceramic parts for medical applications, preferably implants, are intended to be provided into the porous structures of which fastening means such as screws or nails can be inserted without the porous structure failing or the entire implant being destroyed.

These problems are solved by the ceramic part for medical applications described in claim 1. Preferred embodiments are found in the dependent claims.

Ceramic parts within the meaning of the present invention are medical devices made of ceramics which consist in part or entirely of a ceramic foam. The ceramic foam consists of a ceramic bulk material which has a significant proportion of pores (usually 20 to 95% based on the volume), which may be present so as to be isolated (closed porosity) and/or in a pore network (open porosity). In the following, three examples of parts are given which have different ceramic structures and in which the ceramic foam exhibits different characteristics:

Full foam part: A part of which 100% of the volume consists of ceramic foam. It may, for example, be used as a filler material, having the property of serving as a guide structure for osteoconduction and osseointegration.

3D-structured part: A part which consists both of a porous region and of a significant dense ceramic region. Here, the porous region usually protrudes by more than 1 mm into the part. Examples of this are implants for partial resurfacing, in which the region of the part facing the bone is extensively porous and a narrower region of the part facing the articulation surface comprises a dense ceramic region.

2D-textured part: A part of which the topology of the surface is determined by means of a thin, porous region close to the surface. Here, the porous region protrudes approximately 1 mm into the part, such that the volume proportion of the dense ceramic is greater than in the 3D-structured part. Examples of this are ceramic monoblock joints in which the rear side facing the hip is textured with open pores and the side facing the hip joint ball is made of dense, polished material, preferably ceramic.

According to the invention, parts of which the cross sections are formed by different structures are possible. In this case, these structures may comprise both porous ceramic foam and dense ceramics, with the arrangement of the structures being determined by the application of the parts. As a result, any combination of the above-mentioned structures is conceivable.

In a preferred embodiment, the ceramic parts that consist at least in part of a ceramic foam are ceramic implants, i.e. implants for use in human medicine and implants for use in veterinary medicine for small animals, livestock and domestic animals, particularly preferably implants for applications in human medicine.

Implants preferred according to the invention, which usually have wall thicknesses in the range of from 0.3 to 30 mm, for applications in human medicine are implants for small and large joints, vertebral implants, implants in the field of partial resurfacing, bone replacement materials in the form of filler materials, dental implants, and components or parts of implant systems.

Implants according to the invention for small joints may in particular include implants for the finger joints, toe joints, elbow joints, ankle joints, and wrist joints, and other joints. Implants for large joints include, for example, implants for the hip joint, knee joint, and shoulder joint. The vertebral implants may include cages, total disk replacement (TDR), and vertebral body inserts.

The term “partial resurfacing” within the meaning of the present invention covers partial prostheses that only compensate for local joint/cartilage defects. Usually, these consist of a tribologically optimized, congruent side which faces the joint space and a side which faces the bone and provides anchoring. Partial resurfacing is primarily used in large joints, since these require less (bone) tissue to be removed due to the area of the operation being smaller overall, and as a result, subsequent revision surgery is made considerably easier.

According to the invention, the term “bone replacement material” preferably relates to filler materials, for example in corrective osteotomies, in trauma injuries, in voluminous tissue loss due to tumors, in revisions, i.e. a repeated operation when the first intervention has an unsatisfactory result or when the original implant has a limited service life, resulting in an extended intervention, i.e. a larger tissue region that needs to be resected, for plastic surgery for medically indicated tissue reconstruction due to abnormalities, as well as purely esthetic elective interventions, and for defects in the calvaria or the jawbone and craniofacial bone. It is important here that the porous structures, whether they are on a surface or in the form of a three-dimensional structure, provide special properties relating to their macro- and microstructures in the range of a few mm through to the sub-μm range, since the behavior of cells thus interacting in a biological system can be controlled thereby, for example osseointegration (growing-in of an implant).

The use of structures according to the invention as dental implants relates to the use of in particular pin-shaped implants, which are inserted into the jawbone and osseointegrate therein in order to function as an artificial dental root. Here, the porous region of the dental implant is preferably arranged in the lower region, i.e. the region that contacts the jawbone, while the upper part (head) consists of dense ceramic. Owing to the dense ceramic in the upper region, it is ensured that the interface with the abutment can be sufficiently mechanically loaded. In addition, this dense region allows for a form-fitting connection to the gingiva and therefore prevents germs from penetrating. In order to achieve as great a mechanical stability of the implant as possible, the dense region may extend centrally from the implant head into the porous region.

Ceramic parts made up of structures according to the invention can be used as components in implant systems. Here, when it is inserted to face the bone, the porous region can promote osseointegration.

Owing to the porous region of a structure according to the invention, connection to other non-ceramic materials or substances is also possible or is improved. As a result, it is possible to connect structures according to the invention to other materials, for example by plastics infiltration or bonding. Ceramic and non-ceramic structures can be connected, with a rigid, preferably permanent connection to the non-ceramic material being possible due to the porous region of the ceramic structure. Here, the macrostructure of the porous region of a part is dominated by the pores, the pore size of the porous region of the part being between a few 10 μm and 1 mm, preferably between 50 μm and 1 mm, particularly preferably between 100 and 700 μm. The pore sizes are determined by means of microscope images having a resolution at least of 0.2 pixels/μm and preferably having a resolution in the range of from 0.2 to 1 pixel/μm by software-assisted marking and subsequent calculation of the equivalent diameters. By suitably selecting the pore size, the biological, in particular osseointegrative, properties can be considerably improved.

The porous region more preferably has a porosity of from 20 to 95%, preferably from 55 to 85%. By contrast, the dense region has a residual porosity of max. 5%.

For 3D-structured parts, the porosity is preferably a predominantly open porosity which forms an interconnecting pore network, with at least 60%, particularly preferably at least 85%, of the porosity being open porosities.

Owing to the interconnecting pore network having the above-mentioned pore sizes, it is possible for the osseointegration to also progress from the cut pores close to the surface into deeper pores. The bone can grow in to depths of over 0.5 mm up to 5 mm. At the same time, by the bone growing in more deeply, it is achieved that the implant and the surrounding tissue or bone mechanically interlock through undercut pores.

In addition, the open porosities allow for a nutrient supply by diffusion processes in the extracellular fluid. Furthermore, in the porous region of the part, in particular of the implant according to the invention, micromechanical expansion and thus hydrodynamic circulation processes may occur under mechanical loading by means of its reduced modulus of elasticity (the modulus of elasticity of the ceramic foam is approximately 15%, preferably 10% of the modulus of elasticity of the ceramic bulk material).

These properties of the ceramic parts, in particular the implants, can be implemented highly effectively using foaming methods in which defined pore structures are produced in principle on the basis of foaming agents or blowing agents in a ceramic slip.

The use of a foaming method is also advantageous to the extent that, if the process is carried out correctly, it can be implemented without any significant additional effort in comparison with known types of ceramic slip preparation. For example, no additional shaping structures are required, such as organic balls made of cellulose, fiber structures, or polyurethane foam structures, which are impregnated in specially prepared ceramic slips and then have to be burnt out again in the subsequent manufacturing process (porosification processes, template burnout or conversion, etc.).

The ceramic material for the ceramic part according to the invention may be selected from known and commercially available (ceramic) materials, on the condition that the ceramic material is biocompatible and has lower corrosion behavior and lower ion-release rates in the body than calcium phosphate, e.g. hydroxyapatite (HA) and tricalcium phosphate (TCP), or metals and alloys.

The regions of the ceramic part that may be present, i.e. the porous region made of ceramic foam and the dense region, may consist of the same or a different ceramic material.

Preferred ceramic materials, i.e. also the starting powders for producing the part according to the invention, are oxide-ceramic materials, for example based on alumina or zirconia, or non-oxide-ceramic materials, for example based on silicon nitride or silicon carbide. The basic requirement of the material is its biocompatibility, i.e. that it must not cause any negative reactions in the body. Specifically, a biological evaluation e.g. in accordance with DIN EN ISO 10993 (version: 2010-04) needs to be made for the device.

In a preferred embodiment, the ceramic material is a material made of the mixed-oxide system Al₂O₃—ZrO₂, in particular zirconia toughened alumina (ZTA) ceramics, or ceramic composite materials in which zirconia constitutes the volume-dominant phase, with chemical stabilizers or dispersoids in the form of further metal oxides or mixed oxides also being added to these systems depending on the dominant phase.

Examples of ZTA ceramics in which alumina constitutes the volume-dominant phase are:

A ceramic material which consists of 60 to 98 vol. % of an alumina/chromium oxide mixed crystal in the form of a matrix material that may contain 0.8 to 32.9 vol. % of one or more other mixed crystals, selected from mixed crystals according to one of the general formulas Lao₉Al_{11.76-x}CrₓO₁₉, Me¹Al₁₁₋ₓCrₓO₁₇, Me²Al₁₂₋ₓCrₓO₁₉, Me²Al₁₂₋ₓCrₓO₁₉ or Me³Al₁₁₋ₓCrₓO₁₈, with Me1 representing an alkali metal, Me² representing an alkaline earth metal, Me²′ representing cadmium, lead or mercury and Mea representing a rare earth metal oxide, and x representing a value of from 0.0007 to 0.045, and consists of 2 to 40 vol. % of zirconium dioxide embedded in the matrix material, which may contain, as stabilizing oxides, greater than 10 to 15 mol. % of one or more oxides of cerium, praseodymium, and terbium, and/or 0.2 to 3.5 mol. % yttrium oxide, based on the mixture of zirconium dioxide and stabilizing oxides.

A ceramic material made up of alumina in the form of a ceramic matrix having zirconia dispersed therein and optionally other aggregates or phases, with the alumina proportion being at least 65 vol. % and the zirconia proportion being 10 to 35 vol. %, the zirconia being present in the tetragonal phase in a proportion of 80 to 99%, preferably 90 to 99%, based on the total zirconia content, and the stabilization of the tetragonal phase of the zirconia taking place predominantly mechanically rather than chemically, the total content of chemical stabilizers being <0.2 mol. %, with preferably no chemical stabilizers being used. This material preferably contains another dispersoid phase, the volume fraction of the dispersoids forming the dispersoid phase being up to 10 vol. %, preferably 2 to 8 vol. %, particularly preferably 3 to 6 vol. %. In principle, according to the invention, all substances that are chemically stable, do not dissolve in the alumina or zirconia during the production of the composite material by sintering at high temperatures, and allow micro-deformations at a microscopic level due to its crystal structure can be used as dispersoids. According to the invention, it is possible to both add dispersoids and to form the dispersoids in situ when producing the composite material according to the invention. Examples of dispersoids that are suitable according to the invention are strontium aluminate (SrAl₁₂O₁₆) or lanthanum aluminate (LaAl₁₁O₁₈).

An example of ceramic composite materials in which zirconia constitutes the volume-dominant phase is a ceramic material, a ceramic zirconia matrix, and at least one secondary phase dispersed therein, the zirconia matrix forming a proportion of at least 51 vol. % of the composite material, and the secondary phase forming a proportion of 1 to 49 vol. % of the composite material, the zirconia being present in the tetragonal phase in a proportion of 90 to 99%, preferably 95 to 99%, based on the total zirconia content, and Y2O3, CeO2, Gd2O3, Sm2O3 and/or Er2O3 being contained as chemical stabilizers, the total content of chemical stabilizers being <12 mol. % based on the total zirconia content, and the secondary phase being selected from one or more of the following compounds: strontium hexaaluminate aluminate (SrAl₁₂O₁₆), lanthanum aluminate (LaAl₁₁O₁₈), hydroxyapatite (Ca₁₀(PO₄)₆(OH)₂), fluorapatite Ca₁₀(PO₄)₆F₂), tricalcium phosphate (Ca₃(PO₄)₂), spinel (MgAl₂O₄), alumina (Al₂O₃), yttrium aluminum garnet (Y₃AL₆O₁₂), mullite (Al₆Si₂O₁₃), zircon (ZrSiO₄), quartz (SiO₂), talc (Mg₃Si₄O₁₀(OH)₂), kaolinite (Al₂Si₂O₆(OH)₄), pyrophyllite (Al₂Si₄O₁₀(OH)₂), potassium feldspar (KAISi₃O₈), leucite (KAISi₂O₆) and lithium metasilicate (Li₂SiO₃); strontium hexaaluminate, lanthanum aluminate, hydroxyapatite, fluorapatite, spinel, alumina, and zircon are preferred, and strontium hexaaluminate is particularly preferred.

The average particle size (D50) of the ceramic starting powder can be determined by laser diffraction and according to the invention is preferably in the range of from 0.01 to 50 μm, particularly preferably in the range of from 0.1 to 5 μm.

The particle size in the sintered structure is usually in a similar range of from 0.01 to 50 μm or particularly preferably in the range of from 0.1 to 5 μm in the structure, determined by means of the linear intercept technique in accordance with DIN EN ISO 13383-1 (2016-11).

The ceramic part for medical applications according to the invention consists at least of a porous region and optionally a dense region, the porous region that consists of a ceramic foam preferably having a density in the range of from 0.5 to 2.5 g/cm³, particularly preferably 0.8 to 1.8 g/cm³. The strength of the porous region of the part is preferably in the range of from 5 to 300 MPa, particularly preferably in the range of from 20 to 150 MPa.

The thermal conductivity of the ceramic part is preferably <10 W/km and is thus in a similar range to that of the thermal conductivity of natural tissue. As a result, altered sensitivity to cold and heat due to the use of an implant is reduced for the user or patient, and is preferably completely eliminated.

By using a structure according to the invention that comprises a ceramic foam, the behavior of this structure is significantly altered. In the event of local, high loads, predominantly under pressure, a locally restricted defect therefore occurs, rather than a catastrophic failure of the entire implant. The local damage manifests in the form of fractures in the pore webs and is restricted to the region containing the porous foam. Here, the cracks are prevented from propagating more widely since this material has a low fracture toughness (<1 MPa). It contains pores, which counteract cracks from continually propagating to new boundaries. Owing to this locally restricted material behavior, the material of the porous region becomes compacted, with it being possible for deformation energy to be dissipated and applied stresses to also be distributed and relieved thereby.

This material behavior of a part according to the invention allows for machining methods, e.g. drilling, nailing, screwing, rasping, or abrasive cutting. This makes it possible to fix a part according to the invention in position using fastening means such as screws, nails, pins, etc. These fastening means can be inserted into the region formed by the porous ceramic foam without the part being damaged, which impairs the use thereof.

As a result, the part according to the invention, in particular the porous region made of the ceramic foam, not only encourages the natural tissue to grow in, but also contributes to the fixing before and during the operation, i.e. a connection to the body or other implant material is made possible. The ceramic part of the present invention and its porous region preferably can be screwed, i.e. screws can be inserted, can be nailed, i.e. it is possible to tap in or press in nails, and can be drilled, i.e. holes can be made, as a result of which other form-fitting and/or force-locking connections (e.g. by pins) and stitching are also made possible. The above-mentioned fixing means may have a diameter of up to 5 mm, preferably of up to 3 mm.

Furthermore, the ceramic component and its porous region can also be bonded and can be welded (Bone Welding®). Both in bonding and in Bone Welding®, the porosity of the part according to the invention and its porous region is advantageous, since the implant can be infiltrated by the process material (>0.5 mm deep) and is then also mechanically connected thereto or interlocked therewith, in addition to the chemical bond. As a result, connections to other materials, for example non-ceramic materials such as plastics materials and metals, are also made possible. The different methods for joining the different materials can be carried out within applications, for example during insertion as part of an operation, or separately therefrom, in advance, when producing a component or a part of a system.

1. Ceramic part for medical applications which consists of a porous region and optionally a dense region, wherein the porous region consists of a ceramic foam being formed by an oxide-ceramic material or a non-oxide-ceramic material.

2. Ceramic part for medical applications according to claim 1, wherein the ceramic foam is selected from the Al₂O₃—ZrO₂ mixed-oxide system or ceramic composite materials in which zirconia constitutes the volume-dominant phase.

3. Ceramic part for medical applications according to claim 1, wherein a pore size of the porous region is between a few 10 μm and 1 mm.

4. Ceramic part for medical applications according to claim 1, wherein the porous region has a porosity of from 20 to 95%.

5. Ceramic part for medical applications according to claim 1, wherein the ceramic part is an implant.

6. Ceramic part for medical applications according to claim 5, wherein fastening means can be inserted into the porous region of the implant.

7. Ceramic part for medical applications according to claim 6, wherein the fastening means include screws, pins, and nails.

8. Ceramic part for medical applications according to claim 6, wherein the fastening means have a diameter of up to 5 mm.

9. Ceramic part for medical applications according to claim 5, wherein the porous region can be machined.

10. Ceramic part for medical applications according to claim 9, wherein the machining is carried out by grinding and/or drilling and/or nailing and/or screwing and/or pressing.

11. Ceramic part for medical applications according to claim 1, wherein the porous region can be connected to a non-ceramic material.

12. Ceramic part for medical applications according to claim 11, wherein the porous region and the non-ceramic material are connected by plastics infiltration and/or by bonding.

13. Use of the ceramic part according to claim 1 for implants for applications in human medicine or veterinary medicine.

14. Use of the ceramic part according to claim 13 for medical applications as an implant that has a component size having wall thicknesses of from 0.3 to 30 mm.

15. Use of the ceramic part according to claim 13 for medical applications as a vertebral implant and/or in the field of partial resurfacing and/or as a bone replacement material.

16. Ceramic part for medical applications according to claim 3, wherein the pore size of the porous region is between 50 μm and 1 mm.

17. Ceramic part for medical applications according to claim 16, wherein the pore size of the porous region is between 100 and 700 μm.

18. Ceramic part for medical applications according to claim 4, wherein the porous region has a porosity of from 55 to 85%.
